(19) United States
(12) Patent Application Publication
(10) Pub. No.: US 2010/0222843 A1
(43) Pub. Date: Sep. 2, 2010

(54) STIMULATION DEVICE

(76) Inventors: Peter Tass, Munchen (DE); Volker Sturm, Meckesheim (DE)

Correspondence Address: KF ROSS PC, 5683 RIVERDALE AVENUE, SUITE 203 BOX 900, BRONX, NY, 10471-0900, US

(21) Appl. No.: 12/597,918

(22) PCT Filed: May 6, 2008
(86) PCT No.: PCT/DE08/00763

§ 371 Date: Oct. 28, 2009

(30) Foreign Application Priority Data
May 16, 2007 (DE) 10 2007 022 960.9

(51) Int. Cl. A61N 1/36

(52) U.S. Cl. 607/58; 607/72; 607/62

(57) ABSTRACT

The invention refers to a device comprising a generator unit for generating stimulation signals and a stimulation unit connected to the generator unit, wherein the stimulation unit is designed to stimulate nerve cells in the nucleus accumbens and/or in the amygdaloid nucleus and/or in the fasciculus medialis telencephali and/or in pathways made of dopaminergic areas of the mesencephalon leading to the nucleus accumbens or the amygdaloid nucleus and/or in fiber bundles linking the nucleus accumbens and the amygdaloid nucleus using the stimulation signals.

The invention relates to a stimulation device and a method for neurostimulation, the invention more specifically relating to a stimulation device and a method for neurostimulation in the treatment of alcoholic addiction and other addiction illnesses.

Alcoholism poses one of the most serious socio-medical problems in developed nations. In the Federal Republic of Germany alone, leading addiction risk estimates cite 2.5 to 3 million alcoholics classified as addicts, topped by roughly 6 million people who endanger their health by excessive drinking which, although prevalent in all walks of life, mainly affects men more than women. The costs associated therewith directly and indirectly for its treatment and the knock-on costs due to incapacity to work and accidents involving alcoholism are estimated to total at least 20 billion Euro every year in Germany alone. Roughly half of all serious crimes such as murder, manslaughter, assault or rape are committed under the influence of alcohol. More than 40,000 deaths every year in Germany are the result of excessive alcohol consumption. On an average, alcoholics have a life span 15% less than is normal for the population.

Clinically the most relevant results of alcohol addiction are a significant reduction in life expectancy due to excessive drinking causing accidents, suicides, cirrhosis of the liver, chronic pancreatis and cardiomyopathy, as well as permanent resultant harm, especially to the nervous system, such as polyneuropathy, cerebral atrophy, serious cognitive illnesses and personality changes.

The same applies also to other forms of addiction, e.g. heroin or multiple drug addiction.

Hitherto, alcoholism and other forms of addiction were treated by inpatient detoxification, focusing on a qualified withdrawal with subsequent full or partial inpatient treatment to break the habit lasting 4 to 6 weeks, involving a variety of psychotherapeutic methods, programs geared to preventing a relapse and self-help groups, as well as a pharmacological recurrence prophylaxis.

However, even with an optimum combination of the above-described conservative, i.e. non-operative methods, only 40 to 70% of the addicts show a long-term stable improvement. Even after successful withdrawal after having broken direct physical dependence, the psychic dependence still persists, craving being triggered by e.g. those weaned of the habit coming into contact with alcoholics or drug addicts or revisiting the scene of their former addiction.

It is against this backdrop that the following cites a device as it reads from claim 1, use of the device claimed in claim 1 as set forth in claim 10, use of a neurostimulator as it reads from claim 12 as well as a method as set forth in claim 14 whilst advantageous further embodiments and aspects of the invention read from the sub-claims.

In accordance with one aspect of the invention a device comprises a generator unit for generating stimulation signals and a stimulation unit connected to the generator unit. With the stimulation signals the stimulation unit stimulates nerve cells in the nucleus accumbens and/or in the amygdaloid nucleus and/or in the fasciculus medialis telencephali and/or in pathways made of dopaminergic areas of the mesencephalon leading to the nucleus accumbens or the amygdaloid nucleus and/or in fiber bundles linking the nucleus accumbens and the amygdaloid nucleus using the stimulation signals.

In another aspect of the invention the device can be put to use for the treatment of addiction illnesses.

In yet another aspect of the invention a neurostimulator is put to use for the treatment of addiction illnesses.

The invention will now be detailed by way of example with reference to the drawings in which:

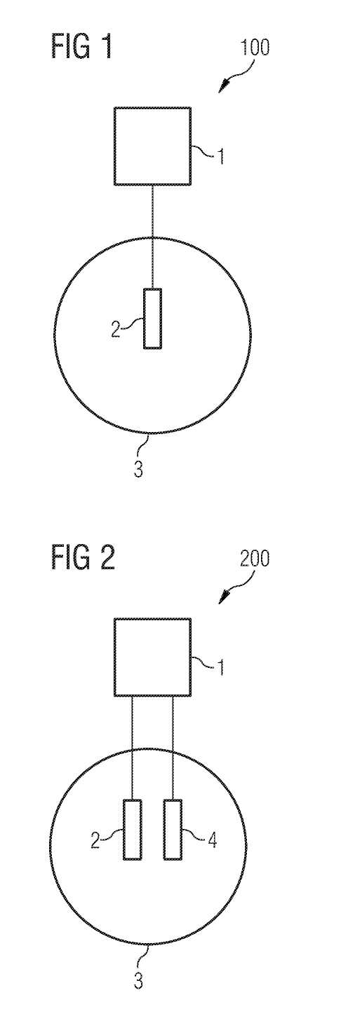

FIG. 1 is a diagrammatic view of a device 100 in accordance with one example embodiment;

FIG. 2 is a diagrammatic view of a device 200 in accordance with a further example embodiment;

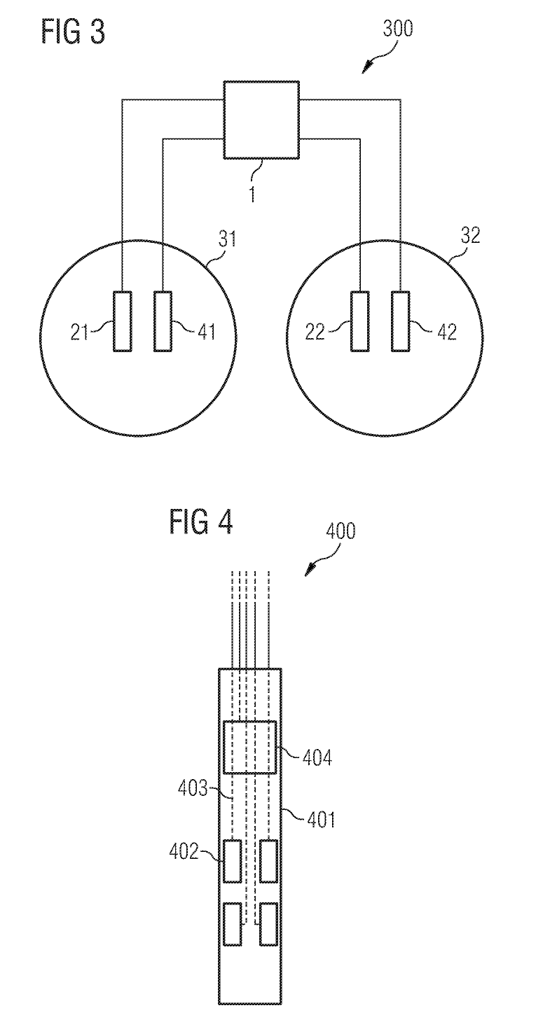

FIG. 3 is a diagrammatic view of a device 300 in accordance with a further example embodiment;

FIG. 4 is a diagrammatic view of a stimulation/detection electrode 400;

FIG. 5 is a diagrammatic view of a high-frequency continuous stimulation;

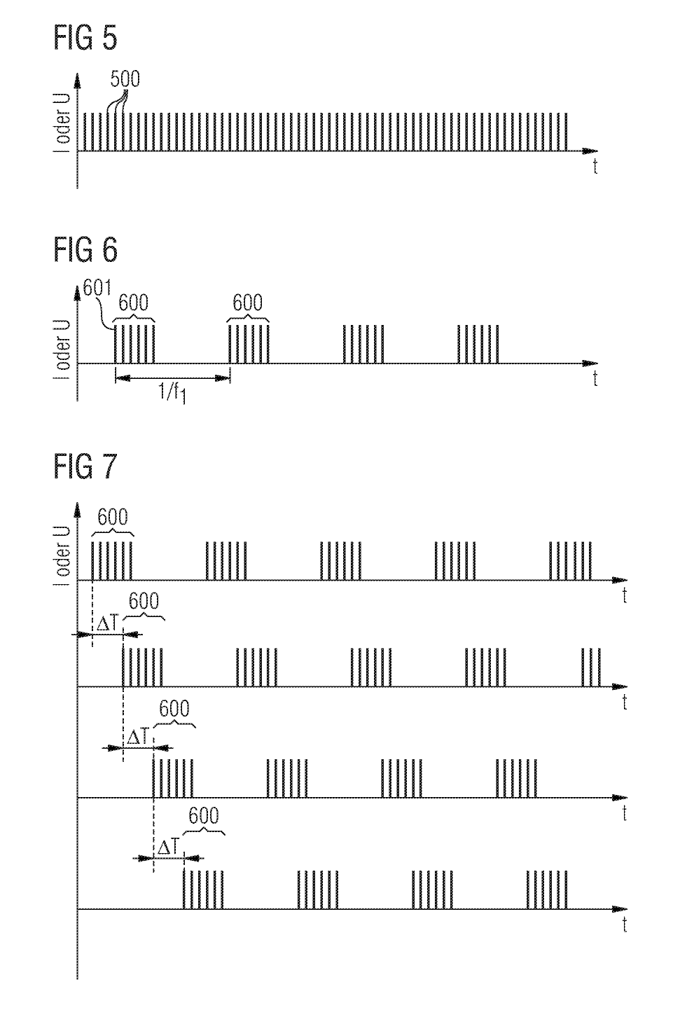

FIG. 6 is a diagrammatic view of a pulse train sequence 600 applied by means of a stimulation contact area;

FIG. 7 is a diagrammatic view of pulse train sequences 600 applied by means of a plurality of stimulation contact areas;

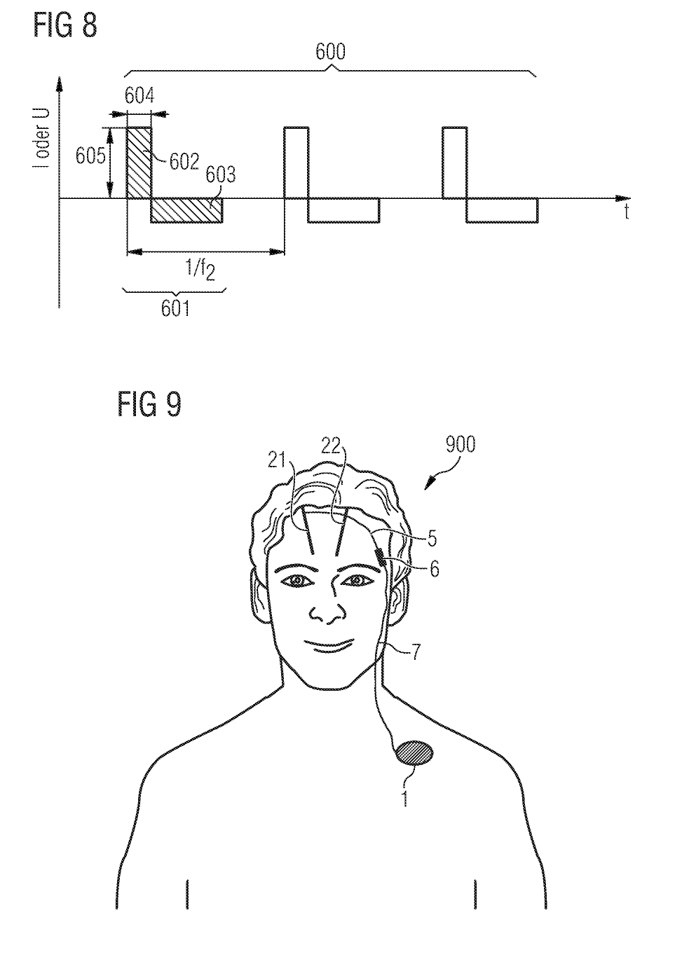

FIG. 8 is a diagrammatic view of a pulse trains 600, and

FIG. 9 is a diagrammatic view of a device 900 in accordance with yet a further example embodiment.

Referring now to FIG. 1 there is illustrated a diagrammatic view of a device 100. The device 100 contains a generator unit 1 and a stimulation unit 2 connected to the generator unit 1. During operation of the device 100 the generator unit 1 generates stimulation signals which are fed into the stimulation unit 2 and used by the stimulation unit 2 to stimulate brain cells. The device 100 is suitable or designed to stimulate, by means of the stimulation unit 2, brain cells in one or more of the following regions 3 of the brain: the nucleus accumbens, amygdaloid nucleus, fasciculus medialis telencephali, pathways made of dopaminergic areas of the mesencephalon leading to the nucleus accumbens or the amygdaloid nucleus as well as fiber bundles linking the nucleus accumbens and the amygdaloid nucleus.

The device 100 can be put to use especially for the treatment of addiction diseases such as e.g. alcoholism, heroin addition, multiple drug addiction and other forms of narcotic dependence.

Addiction diseases such as e.g. alcoholism are diseases of the brain caused by a disorder of bioelectric communication of specifically intercircuited neuronal populations. Functional imaging experiments carried out on animals and diseased patients show that alcohol, like all addiction substances, has a massive effect on the rewarding system of the brain, playing a central role in this respect being the mesolimbic dopaminergic system with neurons originating in the ventral tegmental area (VTA) of the mesencephalon and projection areas in the nucleus accumbens, septum and the amygdaloid nucleus. Taking narcotics triggers a massive activation of the nucleus accumbens, particularly by dopamine release which also especially influences the amygdaloid nucleus located deep in the temporal lobe likewise playing a central role in addiction diseases. Also affected in addition to the nucleus accumbens and the amygdaloid nucleus are the structures in a close spatial and functional relationship to these nuclei, such as the anterior cingulate and medial prefrontal cortex as well as the hypothalamus. Also important in causing addiction is the so-called corpus striatum closely associated with the nucleus accumbens.

The nerve cell populations of the nucleus accumbens and of the amygdaloid nucleus are very easily excited and feature a high tendency to synchronization. Chronic misuse of alcohol or narcotics results in long-term potentiation and/or the so-called kindling phenomenon, as a result of which the neuronal network responsible as a whole for creating a narcotics dependence generates a persistent abnormal neuronal activity and an abnormal connectivity (network structure) associated therewith. This in turn results in a large number of neurons forming synchronous action potentials, i.e. the neurons involved firing excessively synchronized. Where addiction is involved the mean frequency with which the associated neuronal populations are abnormally rhythmically activated is roughly in the range of 1 to 10 Hz, but may also exceed this range.

Neurostimulation as is now implementable with the aid of the device 100 achieves influencing the neurobiological substrates of the addiction illnesses, electrostimulation inhibiting the symptoms and/or reconfiguring the neuronal networks so that the abnormal neuronal activity no longer occurs or at least with significantly less probability. For this purpose, use is made of—in addition to continuous high-frequency stimulation—especially stimulation methods which deconstruct abnormal synaptic links by desynchronization.

The device 100 can be operated, for example, in an open-loop mode in which the generator unit 1 generates predefined stimulation signals and exports them via the stimulation unit 2 to the target areas in the brain. Referring now to FIG. 2 there is illustrated how the device 100 may also be further configured to form a closed loop system in which it additionally includes a detector unit 4 which detects signals fed back from the nerve cells and relays them to the generator unit 1 which may be induced to generate the stimulation signals as prompted by the signals detected by the detector unit 4. The detector unit 4 may take the form of one or more sensors implanted, for example, in one or more of the above-described target areas. These sensors may be engineered, for example, as electrodes, particularly for detecting neuronal and/or vegetative activity or as acceleration sensors. More specifically, the detector unit 4 makes it possible to detect the physiological activity in the stimulated target area or in an area linked thereto.

As regards how the generator unit 1 cooperates with the detector unit 4 a variety of aspects exists. For instance, stimulation may be implemented by the generator unit 1 “on call” (demand-controlled) by the generator unit 1 “seeing” the existence and/or signs of one or more abnormal features from the signals sensed by the detector unit 4. For example, the amplitude or the magnitude of the neuronal activity may be detected and compared to a predefined critical value. The generator unit 1 may be configured to start stimulation of one or more of the above-described target areas as soon as the predefined critical value is exceeded. As an alternative to controlling the point in time of stimulation by way of the signals detected by the detector unit 4, or in addition thereto, the strength of the stimulation signals, for example, can be tweaked depending on how prominent the abnormal features are. For example, one or more critical values may be predefined, resulting in the generator unit 1 tweaking a certain strength of the stimulation signals when the amplitude or magnitude of the detected signals exceeds a certain critical value.

In addition, it may be provided for that the signals detected by the detector unit 4 are used directly or, where necessary, after one or more steps in processing, as stimulation signals and fed by the generator unit 1 into the stimulation unit 2. For example, the detected signals may be amplified and processed, where necessary, after mathematical calculation (e.g. after mixing of the detected signals) involving at least a time delay and linear and/or non-linear steps and combinations thereof before being fed into at least one stimulation contact area of the stimulation unit 2. In doing so, the calculation mode is selected so that the abnormal neuronal activity is counteracted to likewise cause the stimulation signal to disappear or at least be significantly reduced in its strength with diminishment of the abnormal neuronal activity.

Referring now to FIG. 3 there is illustrated in a diagrammatic view a device 300 as a further embodiment of the device 100 or device 200 as shown in FIG. 1 and FIG. 2 respectively. In addition to the generator unit 1 the device 300 features two stimulation units 21 and 22 located in different target areas 31 and 32 of the brain. These target areas 31 and 32 may be the target areas as cited above. In addition to this, the device 300 may also include further stimulation units located in the same or other target areas. Optionally, the device 300 may comprise detector units 41 and 42 likewise located in the target areas 31 and 32 achieving the above-described closed loop operation of the device 300.

Referring now to FIG. 4 there is illustrated in a diagrammatic view an electrode 400 as may be made use of, for example, as the stimulation unit 2, 21 or 22. The electrode 400 consists of an insulated electrode stem 401 and at least one, for example more than two or more, or more than 8 or 12 stimulation contact areas 402 incorporated in the electrode stem 401. These stimulation contact areas 402 are made of an electrically conductive material, for example a metal in direct electrical contact with the nerve tissue when implanted. Each of the stimulation contact areas 402 can be activated by its own lead 403 which may also be used to lead-off the detected signals. In addition to the stimulation contact areas 402 the electrode 400 comprises a reference electrode 404 having a footprint typically larger than that of the stimulation contact areas 402. The reference electrode 404 finds application in generating reference potentials when stimulating the nerve tissue. As an alternative, one of the stimulation contact areas 402 may be used for this purpose.

The electrode 400 is implanted in the head of the patient so that the stimulation contact areas 402 are sited in the nucleus accumbens or in the amygdaloid nucleus or in the fasciculus medialis telencephali or in pathways made of dopaminergic areas of the mesencephalon leading to the nucleus accumbens or the amygdaloid nucleus or in fiber bundles linking the nucleus accumbens and the amygdaloid nucleus.

The stimulation contact areas 402 are typically arrayed closely spaced, the implanted electrode 400 comprising, for example, at least 2 or at least 8 or at least 12 stimulation contact areas 402 covering a path of 7 to 8 mm in the case of the nucleus accumbens and a path of 1 cm where the amygdaloid nucleus is concerned.

In addition to its function as a stimulation unit 2 the electrode 400 may also function as a detector unit 4, in which case signals are detected via at least one of the contact areas 402.

The contact areas 402 can be connected to the generator unit 1 wired or by wireless telemetry.

It was surprisingly discovered that the abnormal neuronal activity and abnormal connectivity associated therewith in addiction illnesses can be counteracted by electrostimulation of the above-described brain areas, continuous stabilization of healthy functioning of these neuronal populations, even as far as total healing of the addiction illness, now being achievable.

Referring now to FIG. 5 there is illustrated in a diagrammatic view a stimulation method suitable for this purpose, namely high-frequency continuous stabilization in which neuronal populations are stimulated by current or voltage-controlled pulses 500 having a repetition frequency of 50 to 250 Hz, particularly 110 to 140 Hz.

Referring now to FIG. 6 there is illustrated in a diagrammatic view how, unlike high-frequency continuous stimulation, stimulation with short pulse trains 600, each composed of a number of single pulses 601 is preferred. Application of the pulse trains 600 is done to the effect that the stimulated, excessively synchronized active neuronal population is desynchronized in returning to its normal response. The pulse trains 600 are applied e.g. as a sequence of up to 20 pulse trains 600 within which the pulse trains 600 are repeated at a frequency f₁ in the range 0.5 to 50 Hz, more specifically in the range 1 to 10 Hz. Aspects of the pulse trains 600 are detailed further on with reference to FIG. 8.

Referring now to FIG. 7 there is illustrated in a diagrammatic view how the pulse trains 600 are administered via the individual stimulation contact areas with a time delay therebetween, showing the pulse trains 600 applied via four different stimulation contact areas illustrated one below the other. Each start of the sequences of pulse trains 600 is shifted in time by DT.

Where N represents the number of stimulation contact areas the time delay DT between any two stimulation contact areas is preferably in the range of a Nth of the mean period of the abnormal rhythmic activity in the target network. Since the mean frequency of the abnormal rhythmic activity of addiction illnesses is roughly 1 to 10 Hz the time delay DT is in the range 0.1 second/N to 1 second/N, for example. In the most favorable case this permits achieving an instant check as to the abnormal neuronal discharge pattern in the target region, the stimulation achieving above all a long-lasting synaptic restructure in the neuronal populations concerned so that the target areas lose the tendency to generate abnormal neuronal activity via plastic activities.

Referring now to FIG. 8 there is illustrated how the pulse trains 600 may each consist of 1 to 100, more specifically 2 to 10 electric charge-balanced single pulses 601, shown here being one such pulse train 600 made up of three single pulses 601 with a frequency f₂ of between 50 to 250 Hz, more specifically above 100 Hz. The single pulses 601 may be current or voltage controlled pulses composed of a header 602 followed by a pulse component 603 flowing in the opposite direction. The polarity of the two pulse components 602 and 603 may also be the opposite of that as shown in FIG. 8. The duration 604 of the pulse component 602 is in the range 1 ms to 450 ms. The amplitude 605 of the pulse component 602 where current-controlled pulses are involved is in the range 0 mA to 10 mA and where the pulses are voltage-controlled the range is 0 to 16 V. The amplitude of pulse component 603 is less than the amplitude 605 of pulse component 602, whereas the duration of pulse component 603 is longer than the that of the pulse component 602. The pulse components 602 and 603 are ideally dimensioned so that the charge communicated thereby is equally the same for both pulse components 602 and 603, i.e. the shaded areas in FIG. 8 are the same in size, with the result that the charge imported into the brain tissue by a single pulses 601 is just the same as is exported from the brain tissue.

Referring now to FIG. 9 there is illustrated how the rectangular shape of the single pulses 601 is the ideal shape, from which it departs depending on the Q of the electronics generating the single pulses 601.

However, instead of pulsed stimulation signals the generator unit 1 may also generate stimulation signals configured otherwise, e.g. temporal stimulus patterns. It is understood that the shapes and parameters of the signals as described above are merely by way of example and that those as provided for may be quite different.

Referring still to FIG. 9 there is illustrated the device 900 when operated as intended for the treatment of addiction illnesses by neuronal stimulation with the stimulation electrodes 21 and 22 implanted in the brain of a patient. Each of the stimulation electrodes 21 and 22 located on both sides in one or more of the above-described target areas is wired by a lead 5 via a connector 6 and a connecting lead 7 to the generator unit 1. All parts of the device 900 are implanted in the body of the patient, the leads 5 and 7 as well as the connector 6 being implanted under the skin. As an alternative, instead of the generator unit 1 being pectorally implanted as shown in FIG. 9 a smaller generator may be implanted directly in the drilling to reduce the rate of infection in the generator pocket and avoiding breakage of the connecting leads 5 and 7. Where closed-loop stimulation is involved the device 900 also features at least one sensor.

1. A device comprising
generator means for generating stimulation signals and
stimulation means connected to the generator means for stimulating nerve cells in the nucleus accumbens or in the amygdaloid nucleus or in the fasciculus medialis telencephali or in pathways made of dopaminergic areas of the mesencephalon leading to the nucleus accumbens or the amygdaloid nucleus or in fiber bundles linking the nucleus accumbens and the amygdaloid nucleus using the stimulation signals.

2. The device as set forth in claim 1 wherein the stimulation signals are pulse train sequences.

3. The device as set forth in claim 1 wherein the stimulation means comprises at least two stimulation contact areas.

4. The device as set forth in claim 3 wherein stimulation signals applied by at least two of the different stimulation contact areas are shifted in time.

5. The device as set forth in claim 4 wherein with N stimulation contact areas the shift in time between two each stimulation contact areas is 1/(f·N) where f is a frequency in the range 1 to 10 Hz.

6. The device as set forth in claim 1 wherein the device further comprises detector means for detecting nerve cell signals.

7. The device as set forth in claim 6 wherein the generator means generates the stimulation signals as a function of the detected signals.

8. The device as set forth in claim 6 wherein the generator means generates the stimulation signals as a function of comparing the detected signals to a predefined critical value.

9. The device as set forth in claim 6 wherein the generator means uses the detected signals as stimulation signals or further processes the detected signals and uses the further processed detected signals as stimulation signals.

10. Use of the device as set forth in claim 1 for the treatment of an addiction illness.

11. The use as set forth in claim 10 wherein the addiction illness involves alcoholism, heroin addiction, multiple drug addiction or other forms of narcotic dependence.

12. Use of a neurostimulator for the treatment of an addiction illness.

13. The use as set forth in claim 12 wherein the addiction illness involves alcoholism, heroin addiction, multiple drug addiction or other forms of narcotic dependence.

14. A method comprising the steps of:
generating stimulation signals, and
stimulating with the stimulation signals nerve cells in the nucleus accumbens or in the amygdaloid nucleus or in the fasciculus medialis telencephali or in pathways made of dopaminergic areas of the mesencephalon leading to the nucleus accumbens or the amygdaloid nucleus or in fiber bundles linking the nucleus accumbens and the amygdaloid nucleus.

15. The method as set forth in claim 14 wherein the stimulation signals are pulse train sequences.

16. The method as set forth in claim 14 wherein the stimulation signals are applied to at least two locations.

17. The method as set forth in claim 16 wherein stimulation signals applied to different locations are shifted in time.

18. The method as set forth in claim 17 wherein with N stimulated locations the shift in time between two each stimulation contact areas is 1/(f·N) where f is a frequency in the range 1 to 10 Hz.

19. The method as set forth in claim 14 wherein nerve cell signals are detected.

20. The method as set forth in claim 19 wherein the stimulation signals are generated as a function of the detected signals.

21. The method as set forth in claim 19 wherein the stimulation signals are generated as a function of comparing the detected signals to a predefined critical value.

22. The method as set forth in claim 19 wherein the detected signals are used as stimulation signals or the detected signals are further processed and the further processed detected signals used as stimulation signals.

23. The method as set forth in claim 14 wherein the method is employed for the treatment of an addiction illness.

24. The method as set forth in claim 23 wherein the addiction illness involves alcoholism, heroin addiction multiple drug addiction or other forms of narcotic dependence.
